(19) United States
(12) Patent Application Publication
(10) Pub. No.: US 2019/0151266 A1
(43) Pub. Date: May 23, 2019

(54) PHARMACEUTICAL COMPOSITION FOR TREATING OR PREVENTING BURN INJURIES

(71) Applicant: Jong Hyun NAM, Seoul (KR)

(72) Inventor: Jong Hyun NAM, Seoul (KR)

(21) Appl. No.: 16/095,356

(22) PCT Filed: Mar. 6, 2017
(86) PCT No.: PCT/KR2017/002372

§ 371 Date: Oct. 20, 2018

(30) Foreign Application Priority Data
Dec. 9, 2016 (KR) 10-2016-0167811

(51) Int. Cl. A61K 31/19; A61K 36/15

(52) U.S. Cl. CPC A61K 31/19; A61K 36/15; A61K 2236/33; A61K 2236/333; A61K 2236/331

(57) ABSTRACT

The present invention relates to a pharmaceutical composition useful for treating or preventing burn injury and a method of treating or preventing burn injury. The pharmaceutical composition includes, as an effective component, abieta-6,8,11,13-tetraaen-18-oic acid of Formula 1, or a pharmaceutically acceptable salt or solvate thereof:

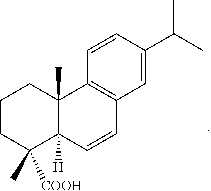 [SMILES: CC(C)c1ccc2c(c1)C=C[C@H]1[C@](C)(C(=O)O)CCC[C@]21C]

### CROSS-REFERENCE TO RELATED APPLICATION

This application is the U.S. national phase of PCT Application No. PCT/KR2017/002372 filed on Mar. 6, 2017, which claims priority to KR Patent Application No. 10 2016 0167811 filed on Dec. 9, 2016, the disclosures of which are incorporated in their entirety by reference herein.

### BACKGROUND

### 1. Field

The present invention relates to a pharmaceutical composition useful for treating or preventing burn injury and a method of treating or preventing burn injury.

### 2. Description of the Related Art

Burns are usually caused by accidents, and can be classified into thermal burns, burns caused by electric currents, chemical burns, and radiation burns according to the causes. The severity of burns can be classified into first-degree, second-degree, third-degree, and fourth-degree burns according to the burned area, depth of burns, the temperature of and the contact time with the object causing burns, and skin condition. In second- or higher-degree burns, scars can be left and hospital cares are required.

For the treatment of burns, it is quite important to heal the early burn wounds as fast as possible or to reduce the burned area. In the initial burn wound dressings, emphasis is placed on the initial treatment to prevent the transition to deep burns by control of infection and inflammation, maintenance of humid environment, and treatment of growth factors or cytokines helping skin regeneration, local use of heparin etc.

If useful therapeutic compounds to treat or prevent burn injury are developed, it would be greatly helpful to treat patients with burns, improve the state, and reduce scars considering the severity of burn injury.

In particular, if the burn injury is not treated quickly, scars may remain, which often prohibits patients from living normal lives. Accordingly, it is quite important to quickly treat the burn injury without side effects or scars.

### SUMMARY

### Technical Purpose

Embodiments of the present invention provide a pharmaceutical composition, which can quickly treat burn injury without side effects.

### Technical Solution

According to an aspect of the present invention, there is provided a pharmaceutical composition comprising, as an effective component, abieta-6,8,11,13-tetraaen-18-oic acid of Formula 1, or a pharmaceutically acceptable salt or solvate thereof

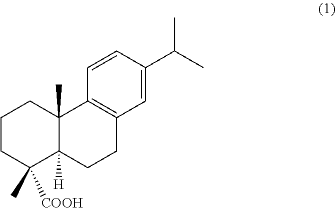 [SMILES: CC(C)c1ccc2c(c1)CC[C@H]1[C@](C)(C(=O)O)CCC[C@]21C]

The pharmaceutical composition according to the present invention uses a naturally derived compound isolated from a carbide extract of pine cone, which is a naturally occurring plant, thereby demonstrating an excellent therapeutic effect against burn injury without side effects.

In particular, according to the present invention, a burn treating composition having excellent pain relief and burn scar preventing effects can be provided.

### BRIEF DESCRIPTION OF THE DRAWINGS

FIG. 1 shows high performance liquid chromatography (HPLC) chromatogram of a compound of Formula 1 isolated from a pine cone carbide extract in accordance with Example 1.

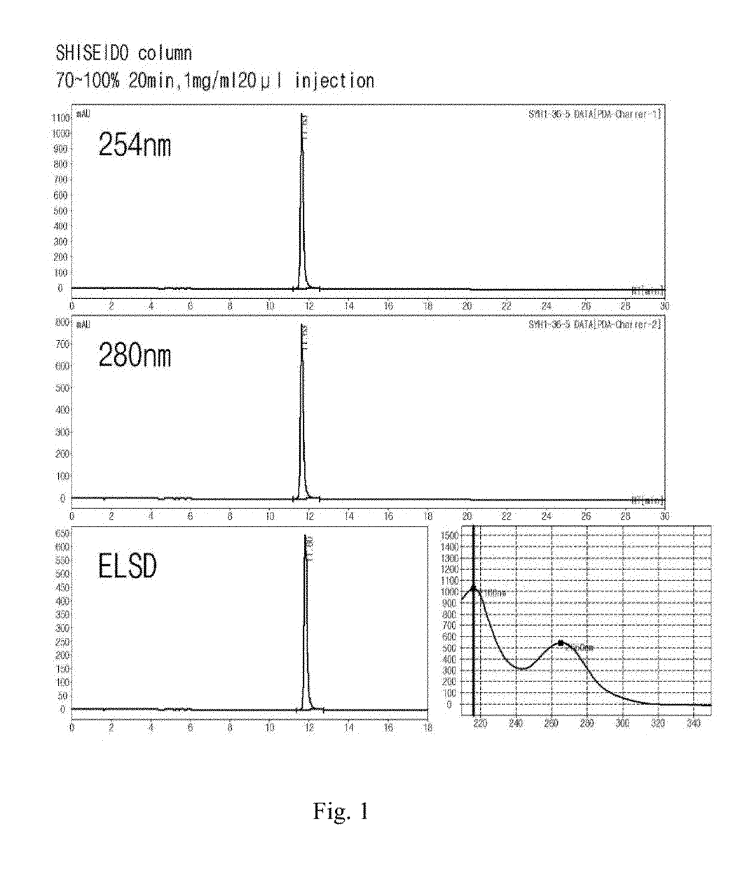

FIG. 2 shows LC/MS spectra of the compound of Formula 1 isolated from a pine cone carbide extract in accordance with Example 1.

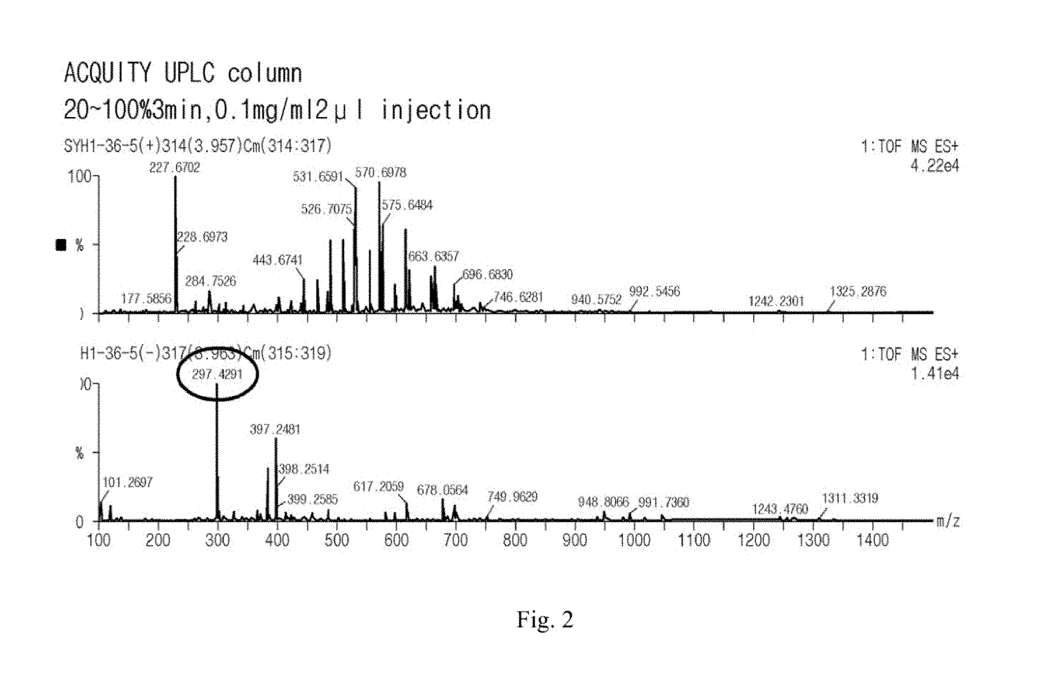

FIG. 3 shows ¹H- and ¹³C-NMR spectra of the compound of Formula 1 isolated from a pine cone carbide extract in accordance with Example 1.

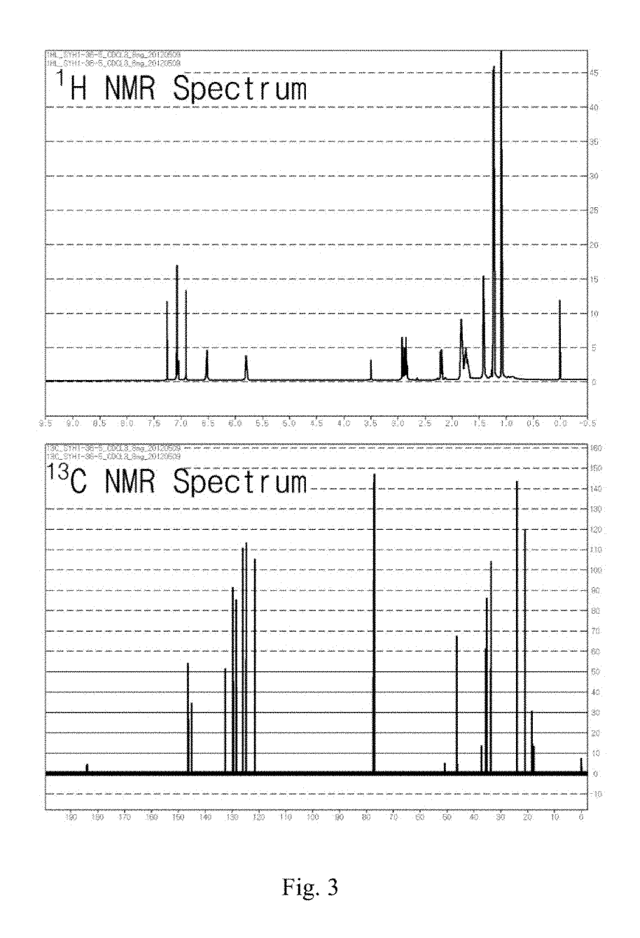

FIG. 4 is a graph showing comparison results of components of a pine cone carbide extract prepared in accordance with Example 2.

FIG. 5 is a graph showing comparison results of components of the pine cone carbide extract prepared in accordance with Example 2, depending on various extraction solvents used.

FIG. 6 is a graph showing comparison results of components of the pine cone carbide extract depending on the sample-to-solvent ratio (W/V) in accordance with Example 2.

FIG. 7 is a graph showing comparison results of components of the pine cone carbide extract depending on the extraction time in accordance with Example 2.

FIG. 8 is a schematic diagram showing an experiment for evaluating the efficacy of the compound of Formula 1, in which A is a stage of planting fluorescence-labeled cells on a stopper equipped surface, B is a state of exposing a cell-free surface by removing the stopper, C is a stage of incubating cells in a cell incubator to induce cell migration, and D is a state of attaching a mask to expose only a site where the stopper was placed and measuring cell migration using a fluorescent microscope or a plate reader.

FIG. 9 is a graph showing the effect of TRPV agonist treatment on an increase in the intracellular calcium level for evaluating pain relief efficacy of the compound of Formula 1.

FIG. 10 is a graph showing a comparative evaluation result of fibroblast proliferation inhibitory efficacy for evaluating a burn treating effect of the compound of Formula 1.

FIG. 11 is a graph of comparing the extract amounts according to the extract solvents.

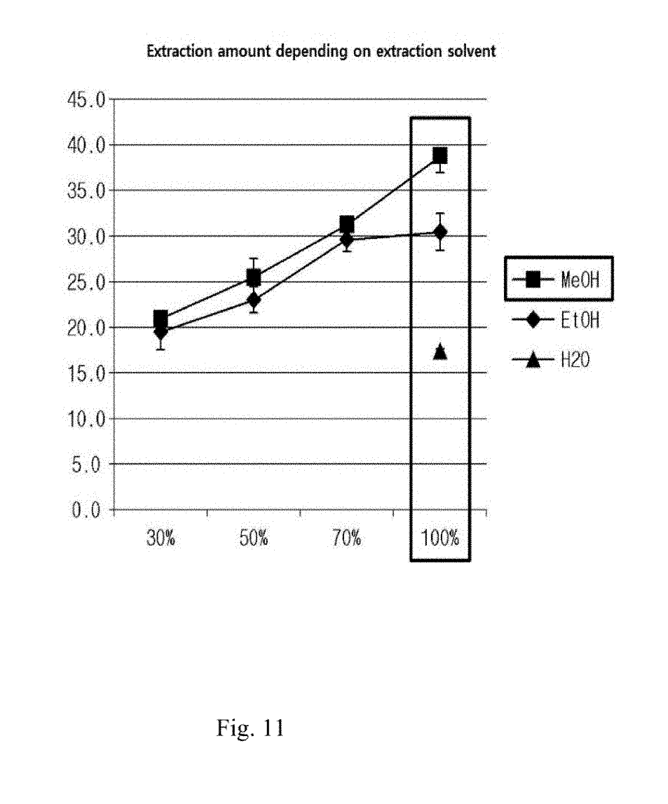

FIG. 12 is a graph of comparing the MeOH extract amount of water extract amount according to the extract time.

### BEST MODE FOR CARRYING OUT THE INVENTION

The present invention will be described more fully hereinafter. This invention may, however, be embodied in many different forms and should not be construed as limited to the embodiments set forth herein.

The present invention provides a pharmaceutical composition useful for treating or preventing burn injury, the pharmaceutical composition comprising, as an effective component, abieta-6,8,11,13-tetraaen-18-oic acid of Formula 1, or a pharmaceutically acceptable salt or solvate thereof:

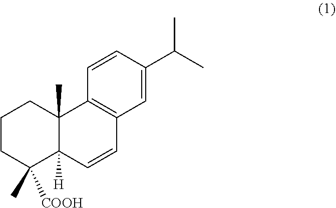 [SMILES: CC(C)c1ccc2c(c1)C=C[C@H]1[C@](C)(C(=O)O)CCC[C@]21C]

wherein H can be all substituted by alkyl groups having 1-10 carbon atoms, and all such substituents are intended to be encompassed within the scope of the present invention.

In the present invention, burns usually refer to the phenomenon that skin cells are destroyed by heat or lead to necrosis. Examples of burns include flame burns caused by fire, scalding burns caused by hot liquid (water, oil, etc.), contact burns caused by contact with hot objects (such as electric irons, rice cookers, etc.), chemical burns caused by strong acids, strong alkalis, sunburns caused by strong ultraviolet light, radiation burns caused by exposure to radiation and X-ray, but are not limited to. Also, the invention of burns can be first degree, second degree, third degree, and fourth degree burns.

The compound of Formula 1 according to the present invention and a pharmaceutically acceptable salt or solvate thereof can be used for treating or preventing burn injury, but the use of the compound of Formula 1 according to the present invention is not limited to specific types and intensity (severity) of the burn.

The compound of Formula 1 according to the present invention can be isolated from a pine cone carbide extract. In addition, the pharmaceutical composition preferably comprises a pine cone carbide extract including the compound of Formula 1. The compound of Formula 1 can demonstrate an excellent burn treating effect without side effects when it is used in the form of a compound isolated from the pine cone carbide extract or a pine cone carbide extract including the compound of Formula 1. However, the preparation method of the compound of Formula 1 is not limited to those disclosed herein.

According to an embodiment of the present invention, the pine cone carbide extract may be prepared by adding water, lower alcohol having 1 to 4 carbon atoms, acetone, ethyl acetate, chloroform, or a mixed solvent thereof to the pine cone carbide. Preferably, the pine cone carbide extract is prepared by adding water, methanol, ethanol, or a mixed solvent thereof to the pine cone carbide. Most preferably, the pine cone carbide extract is prepared by adding 100% methanol as the extraction solvent, which increases the content of an active component in the compound according to the present invention.

According to an embodiment of the present invention, the pine cone carbide extract is preferably prepared by adding the extraction solvent (v) to the pine cone carbide material (w) in a ratio of 1:10 to 1:150 (w/v). If the extraction solvent is used in an amount beyond the ratio stated above, the active component is not sufficiently extracted.

According to an embodiment of the present invention, the pine cone carbide extract is preferably obtained by ultrasonic extraction or reflux extraction. When water is used as the extraction solvent, the reflux extraction is preferably used, and when an organic solvent is used as the extraction solvent, the ultrasonic extraction is preferably used.

The extraction time may be in the range of 10 to 100 minutes, preferably 20 to 40 minutes. Even if the extraction time is prolonged, the amount of the active component extracted is not increased, and if the extraction time is too short, a sufficient amount of the active component is not extracted.

The compound according to the present invention may be administered in the form of a pharmaceutically acceptable salt. The phrase “pharmaceutically acceptable salt” as used herein means salts prepared from non-toxic or low-toxic acids or bases. When the compound according to the present invention contains a relatively acidic functionality, a base-added salt can be obtained by contacting the neutral form of the compound with a sufficient amount of a desired base and an appropriate inert solvent. Examples of the pharmaceutically acceptable base-added salt include, but not limited to, lithium, sodium, potassium, calcium, ammonium, magnesium, organic amino salt, or the like.

The compound of the present invention may include solvates thereof, specifically, hydrates. In addition, the compound of the present invention may include unsolvated forms as well as the solvated forms (e.g., hydrates). The compound of the present invention may exist in crystalline or amorphous forms, and such physical forms are encompassed within the scope of the invention.

The present invention provides a pharmaceutical composition including the compound or a pharmaceutically acceptable salt or solvate thereof, and pharmaceutically acceptable excipients or additives. The compound according to the present invention or the pharmaceutically acceptable salt/solvate thereof may be administered alone or in combination with a convenient carrier or an excipient. The mode of administration may be a single dose or repeated doses.

The pharmaceutical composition according to the present invention may be a solid formulation or a liquid formulation. The solid formulation may take the form of powders, granules, lozenges, capsules, suppositories, and so on, but not limited thereto. The solid formulation may include an excipient, a flavor, a binder, a preservative, a disintegrant, a lubricant, a filler, and so on, but not limited thereto. The liquid formulation may take the form of solutions, such as water or a propylene glycol solution, a suspension, an emulsion, and so on, but not limited thereto. The liquid formulation may be prepared by adding an appropriate coloring agent, a flavor, a stabilizer, a viscosifier, and so on.

The pharmaceutical composition of the present invention may be administered in the form of a formulation suitable for oral administration, injection (e.g., intramuscular injection, intraperitoneal injection, intravenous injection, infusion, subcutaneous injection, or implant), inhalation, intranasal administration, vaginal administration, rectal administration, sublingual administration, transdermal administration, or topical administration, according to the disease to be treated and the condition of a subject, but not limited thereto. According to the route of administration, the pharmaceutical composition of the present invention may be formulated as an appropriate administration unit formulation, which is generally used and non-toxic and includes a pharmaceutically acceptable carrier, additives, and a vehicle. A depot formulation, which can continuously release drugs for a predetermined period of time, may also be included in the scope of the present invention.

The present invention also provides a method for treating or preventing burn injury, including administering to a subject in need of treating or preventing burn injury a therapeutically effective amount of the compound of Formula 1 or a pharmaceutically acceptable salt or solvate thereof.

For the treatment of burn injury, the compound of Formula 1 or a pharmaceutically acceptable salt or solvate thereof may be administered in an amount of about 0.1 mg/kg to about 1000 mg/kg, preferably about 2.5 mg/kg to about 500 mg/kg per day. However, the dose may vary according to the condition of a patient (age, sex, weight, etc.), severity of the condition being treated, the compound used, and so on. If desired, the effective daily dose of the compound may be divided and administered multiple times at appropriate intervals throughout the day.

The present invention will now be further described by means of the following examples, which are put forth so as to provide a complete disclosure but not intended to limit the scope of the present invention.

### Example 1

### Preparation of Pine Cone Carbide Extract and Isolation of the Compound of Formula 1

2.53 kg of pine cone carbide collected from Graeme Co., Ltd., Korea was repeatedly extracted four times at room temperature using 3.5 L of MeOH and was then concentrated to obtain about 92 g of MeOH extract. The obtained MeOH extract was subjected to solvent fractionation three times using each of n-hexane and EtOAc to obtain about 6 g of n-hexane extract and about 45 g of EtOAc extract. Column chromatography (CHCl₃-MeOH, 1:1) using sephadex LH-20 gel was conducted on the EtOAc extract to obtain 5 fractions F1-F5. The fraction F4 was separated using silica gel column (n-hexane-EtOAc, 1:0-0:1) to obtain F4.1-F4.6. Thereafter, the fraction F4.3 was separated using silica gel column (CHCl₃-MeOH, 100:0-98:2) with MPLC (Biorage Isolera ISO-1SV) to obtain F4.3.1-F4.3.4. The fraction F4.3.3 was purified using preparative HPLC (Varian Prostar 210) with YMC-Pack ODA-A column (MeOH-H₂O, 7:3-1:0, 8 mL/min) to isolate the compound of Formula 1.

The isolated compound was subjected to HPLC (Varian 920-LC) and UPLC (Waters ACQUITY UPLC™) to identify its purity, and the molecular weight of the compound was identified using LC/MS (Waters UPLC/q-TOF MS system) and GC/MS (Agilent 7890/5973 MS system). Then, measurement of ¹H-, ¹³C-, ¹H-¹H COSY, HSQC, and HMBC NMR was performed using NMR (Nuclear Magnetic Resonance, Varian system 500 MHz), and the structure of the isolated compound was identified, thereby obtaining the compound of Formula 1:

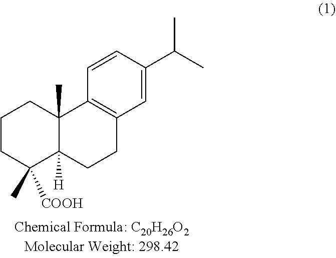 [SMILES: CC(C)c1ccc2c(c1)CC[C@H]1[C@](C)(C(=O)O)CCC[C@]21C]

ESI-MS m/z: 297 [M-H]−. 1H-NMR (500 MHz, CDCl3): δ 1.09 (3H, s, H-20), 1.23 (3H, d, J=2 Hz, H-16), 1.24 (3H, d, J=2 Hz, H-17) 1.42 (3H, s, H-19), 1.68-1.87 (5H, m, H-1a,2,3), 2.21 (1H, d, J=13.5 Hz, H-1b), 2.86 (1H, m, H-15), 2.92 (1H, s, H-5) 5.80 (1H, d, J=10.5 Hz, H-6), 6.53 (1H, d, J=10.5 Hz, H-14), 6.92 (1H, d, J=2 Hz, H-9), 7.08 (2H, m, H-11,12). ¹³C-NMR (125 MHz, CDCl3): δ 17.2 (C-19), 18.4 (C-2), 20.9 (C-20), 24.0 (C-16), 24.0 (C-17), 33.7 (C-15), 35.4 (C-1), 35.8 (C-3), 37.2 (C-10), 46.2 (C-4), 46.6 (C-5), 121.7 (C-11), 124.8 (C-9), 125.8 (C-12), 128.5 (C-14), 129.7 (C-6), 132.6 (C-8), 145.1 (C-9), 145.4 (C-13), 184.0 (C-18).

### Example 2

### Comparison of Pine Cone Carbide Extracts Depending on Extraction Conditions

Each 3 g of pine cone carbides used in Example 1 was taken to be extracted while varying 5 conditions including extraction methods, extraction solvent types, compositions and quantities of extraction solvents used, and extraction time, centrifugation was performed at 4000 rpm to filter a supernatant to then concentrate the supernatant for comparison of extraction amounts, and component analysis was performed using UPLC. Reflux extraction and ultrasonic extraction were used as the extraction methods, and water, ethanol and MeOH solvents were used as the extraction solvents. The compositions of MeOH, EtOH and water as the extraction solvents were varied in the range between 30% and 100%, and the extraction time was varied in the range between 10 to 120 minutes depending on the type of extraction solvent used. Analysis was performed under the same conditions as in Example 1, and the purity of each extract was identified at UV 254 nm.

Extraction methods: reflux extraction/ultrasonic extraction

Extraction solvents used: methanol, ethanol and water

Compositions of extraction solvents: 30%, 50%, 70% and 100%

Quantities of extraction solvents: 30 ml and 100 ml

Extraction time: 10, 30, 60, 90 and 120 minutes

### Comparison by Extraction Method

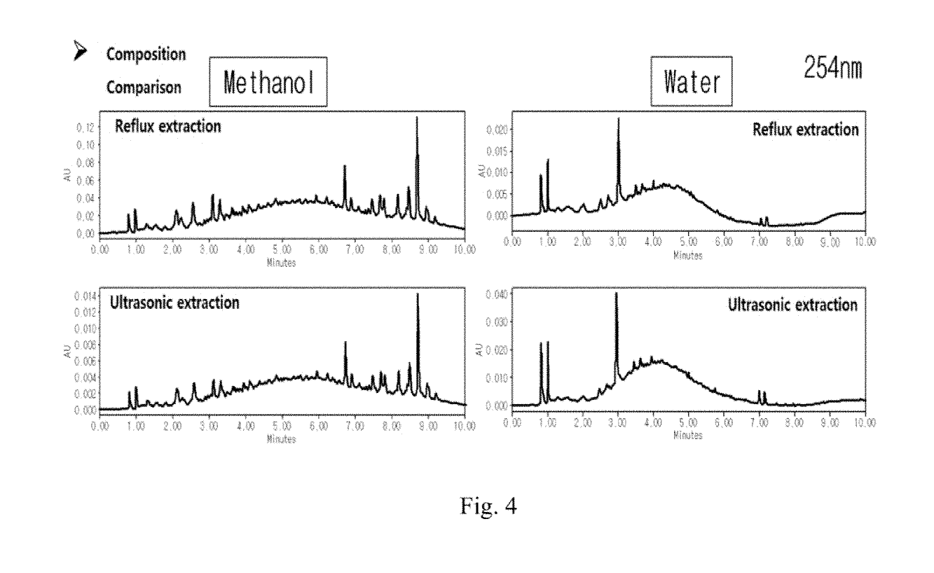

Extraction was performed under the same extraction conditions using each 100 ml of MeOH and water as extraction solvents by ultrasonic extraction and reflux extraction for 30 minutes. When MeOH was used as the extraction solvent, no substantial difference in the extraction amount depending on the extraction method was found. When water was used as the extraction solvent, the extraction amount was larger in the reflux extraction than in the ultrasonic extraction. In the UPLC analysis, no particular difference in the extraction solvent composition depending on the extraction method was found (see FIG. 4).

TABLE 1

| | | Extraction | |
|---|---|---|---|
| | | amount | |
| Solvent | Extraction method | (mg)/1 g | Common conditions |
| MeOH | Reflux extraction | 27.7 | Extraction time: 30 min |
| | Ultrasonic extraction | 27.3 | w/v = 1:33 |
| H₂O | Reflux extraction | 42.0 | |
| | Ultrasonic extraction | 31.6 | |

### Comparison by Extraction Solvent Composition

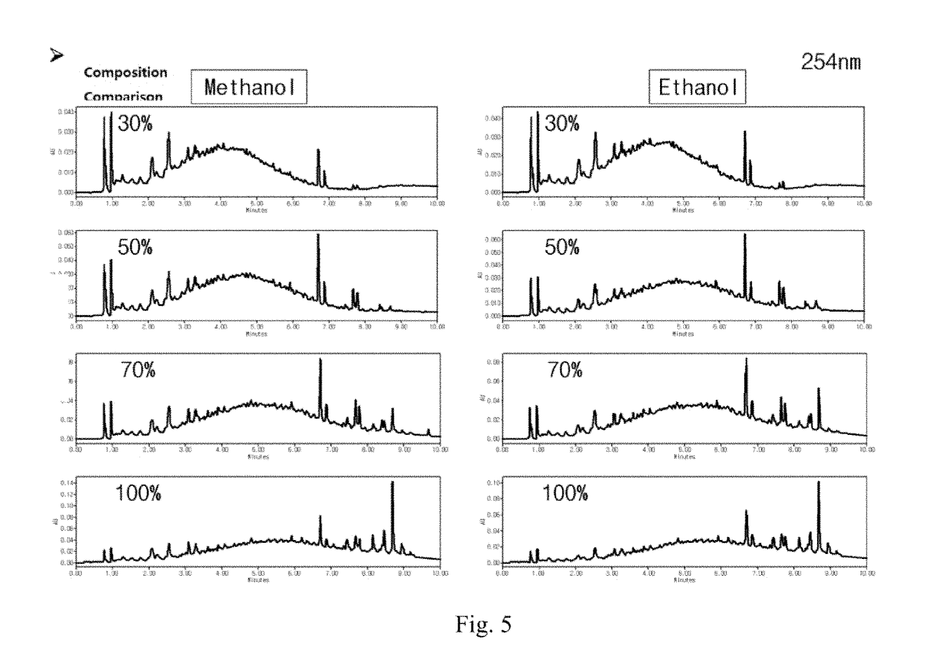

To analyze a difference in the extraction amount depending on the extraction solvent composition, extraction was performed using MeOH, EtOH and water as extraction solvents while varying each composition to 30%, 50%, 70%, and 100%. The extraction was performed under the same conditions using each 30 ml of the extraction solvents by ultrasonic extraction for 30 minutes. When MeOH was used as the extraction solvent, the extraction amount was larger than when EtOH was used under the same conditions. Particularly, when 100% MeOH was used, the extraction efficiency was highest. In the UPLC analysis, no substantial difference in the extraction solvent composition between MeOH and EtOH was found. There was a difference in the content between polar and non-polar components depending on the extraction solvent composition (see FIG. 5).

TABLE 2

| | | Extraction | Common |
|---|---|---|---|
| Solvent | Composition | amount | conditions |
| MeOH | 30% | 7.0 | Ultrasonic |
| | 50% | 8.5 | extraction |
| | 70% | 10.4 | Extraction |
| | 100% | 13.0 | time: 30 min |
| EtOH | 30% | 6.5 | w/v = 1:10 |
| | 50% | 7.7 | |
| | 70% | 9.9 | |
| | 100% | 10.1 | |
| H2O | 100% | 5.8 | |

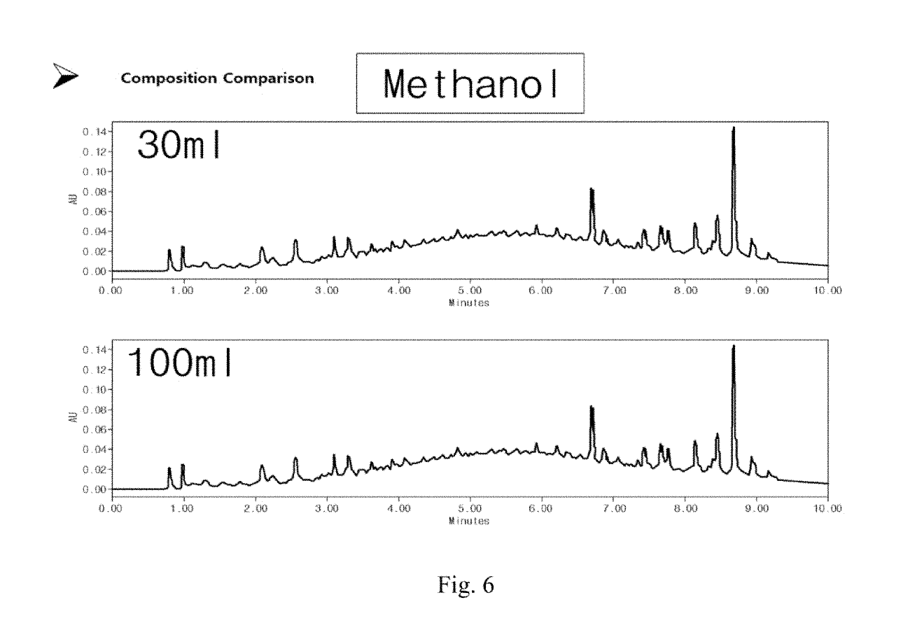

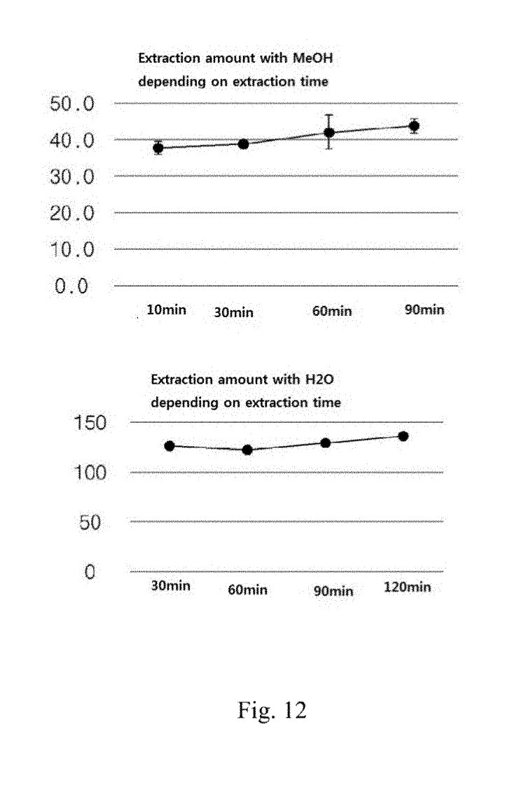

Additionally, differences in the extraction amount depending on the sample-to-solvent ratio (w/v) were compared by performing extraction using MeOH as the extraction solvent. The extraction was performed under the same conditions using the sample-to-solvent ratios (w/v) of 1:10 (30 ml) and 1:30 (100 ml). The extraction result confirmed that the extraction amount was about two times or more when 100 ml MeOH was used than when 30 ml MeOH was used. No component difference was found (see FIG. 6 and FIG. 12).

TABLE 3
Comparison of extraction amounts

| | Extraction | Extraction | |
|---|---|---|---|
| | solvent | amount | |
| Solvent | quantity (ml) | (mg)/1 g | Common conditions |
| MeOH | 30 ml | 13.0 | Ultrasonic extraction |
| | (w/v = 1:10) | | Extraction time: 30 min |
| | 100 ml | 27.3 | |
| | (w/v = 1:33) | | |

### Comparison by Extraction Time

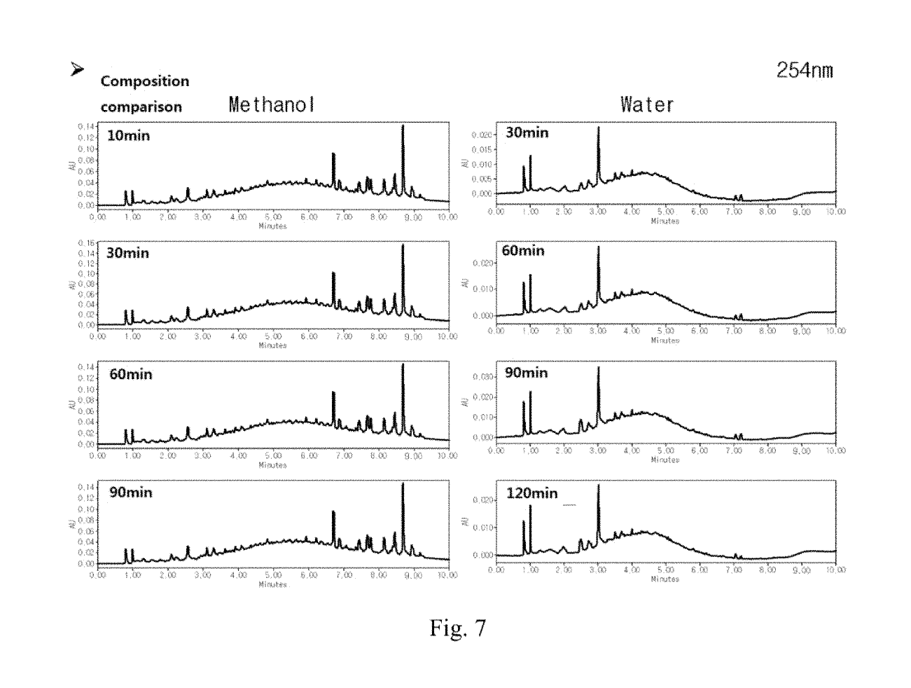

For comparison of extraction amounts depending on the extraction time, experiments were carried out using both extraction methods and suitable extraction solvents corresponding to the extraction methods. The extraction amounts were compared by performing ultrasonic extraction using 30 ml MeOH solvent under four extraction time conditions of 10, 30, 60 and 90 minutes. The extraction amounts and solvent components were compared by performing reflux extraction using 100 ml H2O solvent, which exhibited a relatively high extraction efficiency, under four extraction time conditions of 30, 60, 90 and 120 minutes. All of the extraction conditions, except for the extraction time, were maintained to be the same for both extraction methods. The extraction result showed that the extraction amount was slightly increased according to the increase in the extraction time. Only a slight difference in the extraction amount was found and the difference was considered statistically insignificant. In addition, no particular difference in the extraction solvent composition depending on the extraction time was found (see FIG. 7 and FIG. 12).

TABLE 4
Comparison of extraction amounts

| | | | Extraction | |
|---|---|---|---|---|
| Extraction | | | amount | Common |
| method | Solvent | Time | (mg)/1 g | conditions |
| Ultrasonic | MeOH | 10 min | 12.6 | w/v = 1:10 |
| extraction | | 30 min | 13.0 | |
| | | 60 min | 14.0 | |
| | | 90 min | 14.6 | |
| Reflux | H2O | 30 min | 42.0 | w/v = 1:33 |
| extraction | | 60 min | 40.7 | |
| | | 90 min | 42.9 | |
| | | 120 min | 45.2 | |

As described above, it can be understood that the active component of the pine cone carbide can be extracted with highest efficiency when water and lower alcohol were used.

### Experimental Example 1

### Activity Assay of Compound of Formula 1

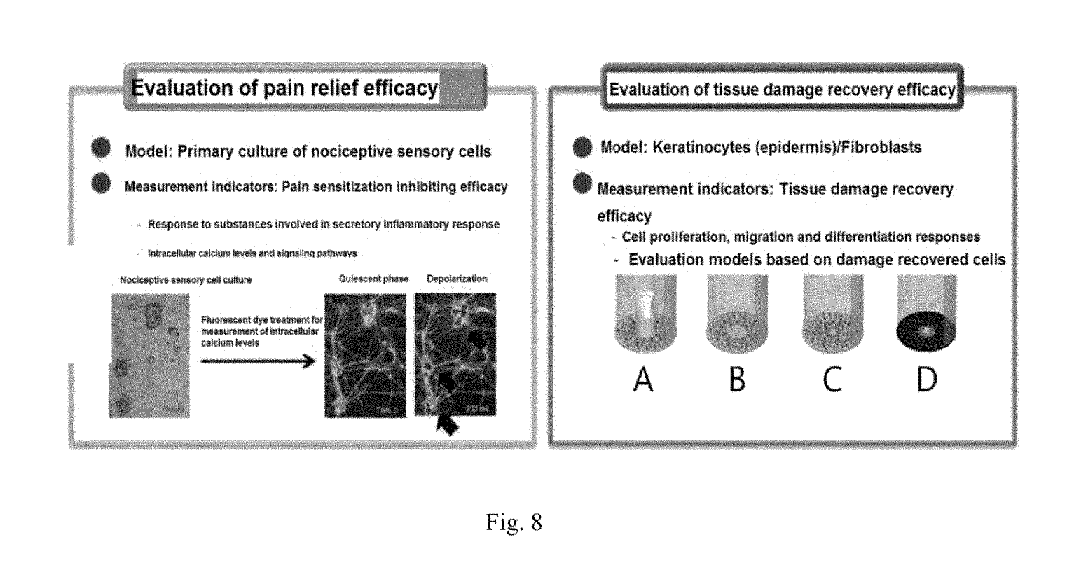

To investigate the activity of the compound of Formula 1 isolated in Example 1, pain relief and tissue damage recovery efficacies were evaluated. The pain relief efficacy was confirmed by identifying pain sensitization inhibiting efficacy, responses to substances involved in secretory inflammatory responses, intracellular calcium levels and signaling pathways from nociceptive sensory cells. The tissue damage recovery efficacy was confirmed by identifying tissue damage recovery, and cell proliferation, migration and differentiation responses from keratinocytes and fibroblasts (see FIG. 8).

### Experimental Example 1-1

### Evaluation of Pain Relief Efficacy

After fl 1 nociceptive sensory cell lines in white rats incubated with the compound of Formula 1 for 24 hours were pretreated, TRPV ion channel activities were measured. The fl 1 cell lines were treated with 2-APB (2-aminoethoxydiphenyl borate) as a pre-activator for TRPV ion channels, and intracellular calcium changes, which are triggered in response to the 2-APB treatment, were evaluated using a Fluo-4-AM fluorescent dye on a real time basis.

It was confirmed that when the fl 1 cell lines were treated with 2-APB as the pre-activator for the TRPV ion channels, a sharp increase in the intracellular calcium level was transiently shown within 20 seconds and the increased intracellular calcium level was reverted to the base level in about 5 minutes.

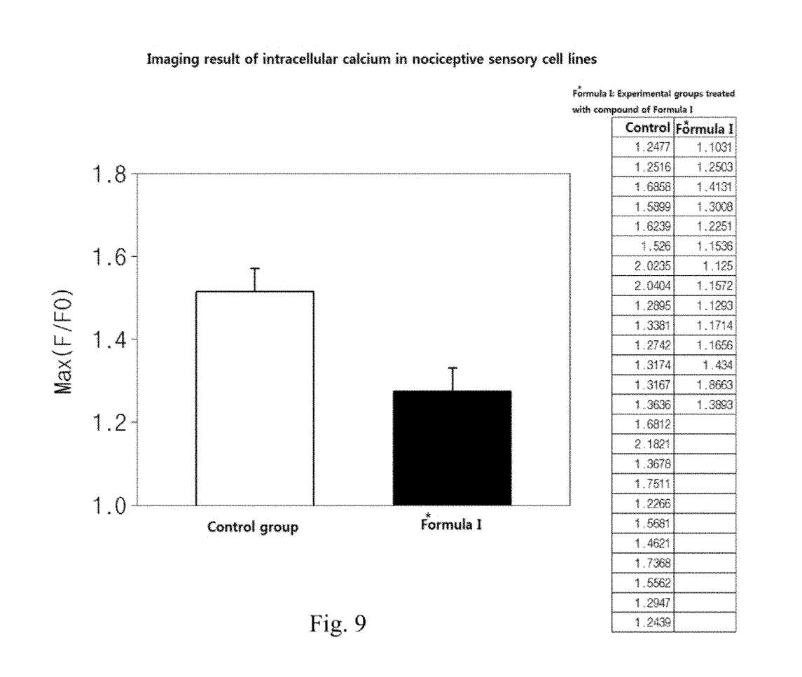

However, when the fl 1 cell lines were pre-treated with the compound of Formula 1 for 24 hours, an increase in the intracellular calcium level induced by 2-APB was strongly inhibited. Presenting that the compound of Formula 1 provided a pain relief efficacy is confirmed in FIG. 9 and the below table of comparison between the experimental groups treated with the compound of Formula 1 and the control groups untreated therewith (see FIG. 9).

| Control | Formula 1 |
|---|---|
| 1.2477 | 1.1031 |
| 1.2516 | 1.2503 |
| 1.6858 | 1.4131 |
| 1.5899 | 1.3008 |
| 1.6239 | 1.2251 |
| 1.526 | 1.1536 |
| 2.0235 | 1.125 |
| 2.0404 | 1.1572 |
| 1.2895 | 1.1293 |
| 1.3381 | 1.1714 |
| 1.2742 | 1.1656 |
| 1.3174 | 1.434 |
| 1.3167 | 1.8663 |
| 1.3636 | 1.3893 |
| 1.6812 | |
| 2.1821 | |
| 1.3678 | |
| 1.7511 | |
| 1.2266 | |
| 1.5681 | |
| 1.4621 | |
| 1.7368 | |
| 1.5562 | |
| 1.2947 | |
| 1.2439 | |

### Experimental Example 1-2

### Evaluation of Inhibitory Efficacy of Fibroblast Cell Proliferation

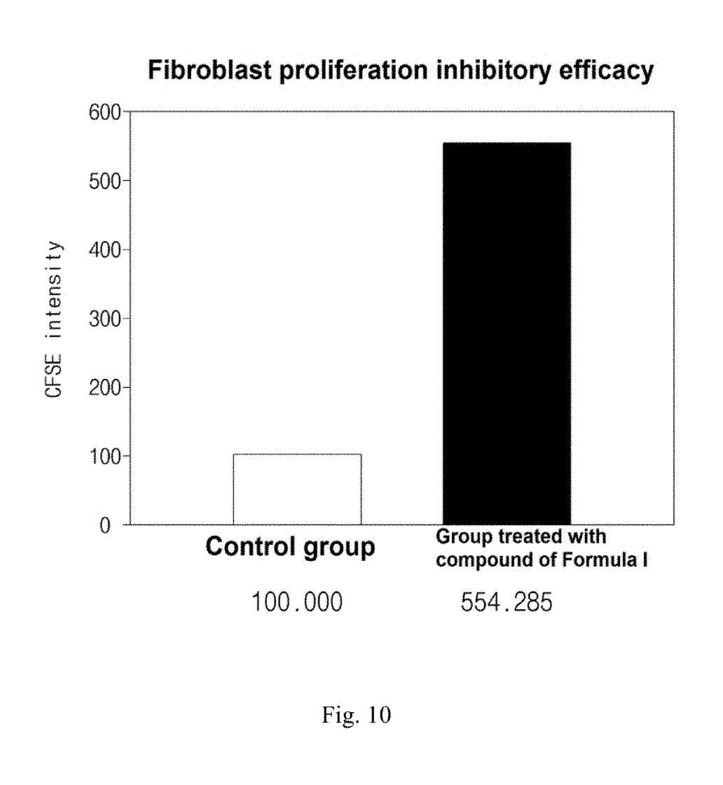

After f11 neuron cell lines were treated with the compound of Formula 1 for 72 hours, the extents of cell differentiation were first compared/evaluated (see FIG. 10). The experimental groups treated with compound of Formula 1 showed improved fibroblast cell proliferation and differentiation inhibitory efficacy, compared to the control groups.

Since fibroblast cell proliferation accompanies excessive collagen synthesis to make up for the dermis damaged by skin injury, scars can be left even after the damage recovery. Therefore, scar prevent or treating efficacy can be exerted by inhibiting the fibroblast proliferation.

1. A pharmaceutical composition useful for treating or preventing burn injury, the pharmaceutical composition comprising, as an effective component, abieta-6,8,11,13-tetraaen-18-oic acid of Formula 1, or a pharmaceutically acceptable salt or solvate thereof:

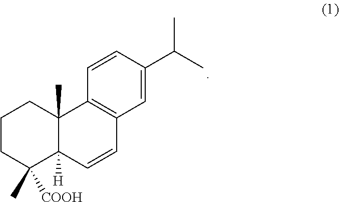 [SMILES: CC(C)c1ccc2c(c1)C=C[C@H]1[C@](C)(C(=O)O)CCC[C@]21C]

2. The pharmaceutical composition of claim 1, wherein the pharmaceutical composition comprises a pine cone carbide extract including the compound of Formula 1.

3. The pharmaceutical composition of claim 2, wherein the pine cone carbide extract is prepared by adding water, lower alcohol having 1 to 4 carbon atoms, acetone, ethyl acetate, chloroform, or a mixed solvent thereof to the pine cone carbide.

4. The pharmaceutical composition of claim 2, wherein the pine cone carbide extract is prepared by adding water, lower alcohol having 1 to 4 carbon atoms, or a mixed solvent thereof to the pine cone carbide.

5. The pharmaceutical composition of claim 2, wherein the pine cone carbide extract is prepared by adding 100% methanol to the pine cone carbide.

6. The pharmaceutical composition of claim 2, wherein the pine cone carbide extract is prepared by adding an extraction solvent to the pine cone carbide in a ratio of 1:1 to 1:5 (w/v).

7. The pharmaceutical composition of claim 2, wherein the pine cone carbide extract is obtained by ultrasonic extraction or reflux extraction.
